(19) United States
(12) Patent Application Publication
(10) Pub. No.: US 2011/0065160 A1
(43) Pub. Date: Mar. 17, 2011

(54) TREATMENT OF EUKARYOTIC CELLULAR BIOMASS

(75) Inventors: Peter Barratt, Overton Hampshire (GB); Jake Spooner, New Milton Hampshire (GB)

(73) Assignee: Shepherd, Samuel, Houston, TX (US)

(21) Appl. No.: 12/745,574

(22) PCT Filed: Nov. 26, 2008
(86) PCT No.: PCT/GB08/03951

§ 371 Date: Nov. 19, 2010

(30) Foreign Application Priority Data
Nov. 30, 2007 (GB) 0723504.7

(51) Int. Cl. C12P 7/10; C12P 7/06; C12P 7/04; C12P 5/02; C12M 1/00

(52) U.S. Cl. 435/165; 435/161; 435/157; 435/167; 435/289.1

(57) ABSTRACT

The application describes a process for treating a eukaryotic cell-derived biomass-containing stream including: (i) passing the stream through a chamber; (ii) pressurizing_the stream; (iii) introducing a gas into the pressurized stream, the gas being soluble within the eukaryotic cell-derived biomass; and (iv) depressurizing the stream to cause the solubilised gas to expand and disrupt the eukaryotic cell-derived biomass. The biomass may be derived from plant or animal materials.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The invention relates to processes and an apparatus for treating eukaryotic cell biomass and derivatives, such as materials derived from wood or animals, and the use of such processes in the production of renewable products, such as ethanol or methane.

2. Description of Related Art Including Information Disclosed Under 37 CFR 1.97 and 37 CFR 1.98

There is increasing interest in the production of fuels or other products from waste materials such as wood chippings or paper, or other waste materials. One problem with such materials is that they need to be broken down to efficiently release compounds such as sugars, which can then be used in other processes, such as fermentation processes. These can then be used to produce useful products such as methane, hydrogen or ethanol, or other fermentation products such as lactic acid, butyric acid or acetone. The biomass, once broken down, may also be used as a source of nutrients to grow organisms such as fungi for food.

WO 2007/059487 discloses a process for treating a micro-organism-containing stream by pressurizing the stream, introducing a feed gas which is soluble within the micro-organisms, and depressurizing to cause the solubilised gas to expand within the micro-organisms and rupture them. Optionally an acid, such as sulfamic acid, nitric acid, phosphoric acid, oxalic acid, hydrochloric acid or sulphuric acid can be added to micro-organisms to reduce the pH below 6.5. The aim of this process is to sterilize sewage sludge and dewater it.

U.S. Pat. No. 5,635,069 discloses mixing waste sludge with an oxide and sulfamic acid, pressurizing the sludge and discharging the pressurized sludge. The oxide and acid are reacted to elevate the temperature of the sludge to between 50° C. and 450° C.

Elevating the pH to at least 9.8 has also been used to treat pathogen-containing sludges (see U.S. Pat. No. 5,868,942). This utilized calcium oxide, ammonia and carbon dioxide and again used pressure. Similarly, U.S. Pat. No. 6,056,880 utilized acid, an oxide and pressure to treat a waste sludge of biological solids.

Fuel products have been produced using sewage sludge mixed with acid and oxide and pressurized. This is then mixed with coal fines and solidified to produce a fuel material.

### BRIEF SUMMARY OF THE INVENTION

The inventors have realized that the principles shown in the prior art for breaking open microbial cells could also be used to assist the breakdown of multi-cellular structures such as wood or animal cells. It could also be used to break down materials derived from such products, such as paper or cotton.

Accordingly, the first aspect of the invention provides a process for treating a eukaryotic cell-derived biomass-containing stream comprising:

(i) passing the stream through a chamber;

(ii) pressurizing the stream;

(iii) introducing a gas into the pressurized stream, the gas being soluble within the eukaryotic cell-derived biomass; and

(iv) depressurizing the stream to cause the solubilised gas to expand and disrupt the eukaryotic cell-derived biomass.

Preferably the stream and gas are kept in the chamber or a subsequent residence chamber for sufficient time for the gas and stream to equilibrate. Typically, this is between 1 and 60 minutes, or 1 and 30 minutes.

Eukaryotic cell-derived biomass may be material still containing eukaryotic cells, such as wood, herbaceous plant material, grass clippings or animal, such as cow, pig, sheep, goat, horse or fish, tissue, and additionally includes material derived from such cells, such as cotton, cellulose and collagen. Such biomass may comprise a mixture of various materials, of both plant and animal origins, such as food waste.

Preferably, the eukaryotic cell-derived biomass comprises plant-derived material. Such plant material preferably comprises cellulose, lignin and/or hemicellulose. The plant-derived material preferably comprises wood chippings, sawdust, paper, herbaceous plant material such as weeds or other plant material from food and non-food plant crops, grass clippings, cotton, hemp and/or flax. The cotton, hemp and/or flax may be in the form of recycled clothing such as cotton-containing clothing or linen.

Alternatively, or additionally, the eukaryotic cell-derived biomass may be obtained from animal material and include proteinaceous animal material, such as collagen, flesh and/or spinal tissue.

The eukaryotic cell-derived biomass-containing stream maybe derived from municipal waste. Such municipal waste may have other materials, such as plastics or metals, removed by techniques known in the art such as sieving, hand sorting or, for example, separated by fluid-dynamic separation, prior to being passed through the chamber. The eukaryotic cell-derived biomass may also comprise food waste.

Preferably, the biomass is broken up, for example, by chopping, shredding or macerating into particles. The physical breakdown of the material assists in increasing the surface area open to the surrounding medium.

The biomass stream may have the moisture content adjusted, for example, by the addition of steam or water or another aqueous liquid, such as downstream process liquors. Typically, the solids content of the biomass is adjusted to within the range 2-50% dry solids by weight. This may be achieved by treating with, for example, steam for 1 minute or, for example, soaking in water for up to, typically, 4 hours. The aqueous liquid may be fresh or recycled water and may be added prior to or after physically breaking down the biomass prior to passing through the chamber.

Preferably, the biomass stream is not sewage, sewage sludge or fecal material.

Preferably, the biomass material has moisture added so that it contains at least 2%, preferably at least 5% dry solids by weight, or at least 10% dry solids by weight.

The biomass stream is passed through a chamber. The chamber is pressurised to above atmospheric pressure. Typically, the atmospheric pressure within the chamber is up to 25 barg (bar gauge), but is typically between 0.5 barg-12 barg, or up to 10 barg or up to 6 barg.

The process can be operated on a batch or continuous basis with pressure being increased gradually or rapidly.

The gas is added into the pressurized stream. Under pressure, the gas dissolves within the moisture of the eukaryotic cell-derived biomass.

The stream is rapidly depressurized to cause the solubilised gas to expand. This rapid expansion results in the expansion of the dissolved gas into bubbles. The gas expands by as much as 1800% upon depressurization. Depressurization may be carried out in, for example, a flash chamber which has a lesser pressure than the pressure within the chamber.

The expansion of the solubilised gas disrupts the eukaryotic cell-derived biomass and increases both the surface area of the material available for downstream processes, and the availability of, for example, sugars or proteins in the stream.

The gas used for pressurization is preferably carbon dioxide. This assists in providing acidification of the stream, which may assist in hydrolyzing the biomass. This may be present in the form of 1-100% CO2 by volume, most preferably 25-100% by volume. Alternative gases include air, nitrogen, methane and mixtures of gases. For example, the gas may be methane-carbon dioxide mixtures formed from the anaerobic digestion of the depressurized stream in a bioreactor.

The gas released from the depressurization step may be recycled and used again.

The breakdown of the biomass can be further increased by treating the stream before and/or during the pressurization step with one or more physical, chemical or biological treatments.

For example, chemical treatment may comprise treating the biomass with wetting agents such as sodium hydroxide, potassium hydroxide, lithium hydroxide, ammonium hydroxide, benzyl trimethylammonium sulphate, zinc chloride, calcium carbonate, sodium carbonate, sulphur dioxide, sulphuric acid or phosphoric acid. Other chemicals include hydrogen peroxide or calcium oxide. Organic solvents, such as methanol, may also be incorporated. Furthermore, detergents may also be incorporated.

U.S. Pat. No. 4,304,649 discloses many of the above agents in the solubilisation of lignocellulosic materials.

Treatment of materials, such as lignocellulosic materials with alkali or acids may also be used (see U.S. Pat. No. 5,515,816). Preferably, alkalis such as sodium hydroxide are used. Mineral acids, such as sulphuric acid and metal alkali hydroxide, may be used.

Carbon dioxide, which is the especially preferred gas, dissolves better under acidic conditions. Moreover, carbon dioxide itself forms an acid in water and assists the process.

Typically, the chemical, including alkali or acid treatments, are contacted with the stream for 1-60 minutes. They are typically added as solubilised salts, where appropriate, to the feed stock biomass material.

Acid treatments may be utilised for longer periods of time, as disclosed in U.S. Pat. No. 4,515,816, which shows that lignocellulosic material may be treated for 5-21 days in dilute aqueous acid at pH 2-3, to induce mild hydrolysis.

Biological materials may also be used, in the form of whole micro-organisms or extracts of micro-organisms to break down and release of carbon-containing feed materials to the production process. Such treatments use intracellular or extracellular enzymes such as peroxidases and chitinases, or organic acids produced on the living micro-organism, such as those used in bioleaching of metals from ores. Living micro-organisms such as Lactobacillus species may be utilized, such as those used in agricultural silage production.

Physical treatments include heating and particle size reduction, by, for example, high-shear mixers or macerators. Most preferably, the physical heating includes the use of steam. Steam has previously been used with physical disintegration methods at temperatures of in excess of 150° C. The pre- or co-treatment using heat with the addition of pressurised carbon dioxide outlined in the invention can be used to reduce the temperatures, pressures and residence times required for steam treatment. Where co-treatment with heat and carbon dioxide is considered, then temperatures in the range of 40-180° C. are preferable.

The depressurized stream is preferably directed towards a bioreactor, for example an anaerobic or aerobic bioreactor. The stream is then digested, for example, utilizing suitable bacteria or enzymes to produce products such as methane, hydrogen, ethanol, lactic acid, butyric acid or acetone. The anaerobic or aerobic fermentation of material is generally known in the art. The residual product of the stream may also be used, for example, as a growth medium for, for example, fungi, plants or micro-organisms. The content of the stream may be varied, for example, by mixing plant waste with animal waste to adjust the amount of carbohydrates and proteins available in the final product.

Preferably, the gas released from the depressurization step is recycled and fed back into the pressurized stream.

Where the depressurized stream is then fermented or otherwise utilized in a bioreactor, such a process often produces a solid product. This solid product itself may be dried and burned to produce heat to either directly or indirectly heat the stream or produce steam for treating the stream prior to or during the pressurization step.

The invention also provides an apparatus comprising an entrance port for receiving a eukaryotic cell-derived biomass containing stream; a port for adding an aqueous liquid to the stream; a chamber for pressurizing the stream, the chamber comprising a port for introducing a gas into the pressurized stream; a depressurization chamber for depressurizing the stream exiting the chamber, and a bioreactor for receiving the depressurized stream.

A residence chamber may be provided after the chamber where the stream and gas can equilibrate, prior to depressurization.

Apparatus for use in the processes of the invention are also provided.

A further aspect of the invention provides an apparatus according to the invention when used in the process according to the invention.

Preferred uses and features of the apparatus may be as defined above.

### BRIEF DESCRIPTION OF THE SEVERAL VIEWS OF THE DRAWINGS

The invention will now be described by way of example only with reference to the following FIGURE.

FIG. 1 shows a flow diagram summarizing a process according to the invention.

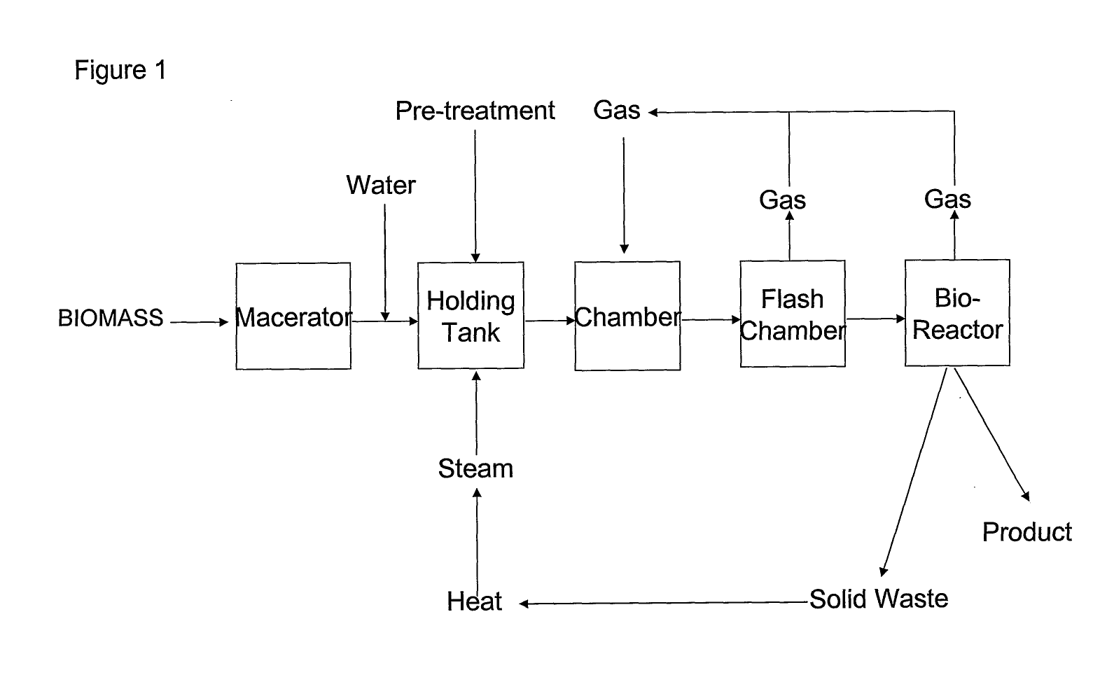

### DETAILED DESCRIPTION OF THE INVENTION

The figure shows a eukaryotic cell-derived biomass-containing stream, which enters the process at an entrance port. The biomass may be, for example, cellulosic material such as wood chippings, paper, sawdust, herbaceous plant material, grass clippings, algae, mixed food materials, cotton, hemp and/or flax. Proteinaceous animal material, such as collagen, flesh and/or spinal tissue may also be used. With respect to the latter material, the advantage of the process is that the process will at least partially sterilize the material, thus reducing the chance that the material contains pathogens. The biomass is passed to a macerator which breaks down the material into smaller components. Where necessary, water or another aqueous fluid, is added to the material in order to raise the moisture content of the material to typically 2-50% by weight dry material. Steam may also be used to increase the moisture content of the material.

The material then typically passes to a holding tank where it may be heat treated and/or pretreated by an acid or other biological treatment as described above. Typically, a wetting agent such as sodium hydroxide is used to solubilise the material if it is a lignocellulosic material. The holding tank may be separate to the chamber where the material is pressurised. Alternatively, the pressurization holding tank may be the same component of the apparatus used in the process. The chamber is pressurized to typically 0.5-25 barg, especially 0.5-12 barg, or 0.5 to 10 barg or 0.5 to 6 barg. A gas, which is typically a carbon dioxide-containing gas, is introduced into the chamber. The gas dissolves within the moisture in the stream.

A residence chamber may be provided where the stream and gas can equilibrate.

On exiting the chamber or residence chamber, the pressurized stream is depressurized, for example, by passing into a flash chamber. This causes the dissolved gas to expand and break down the biomass within the stream. Gas released from the biomass may be collected and recycled to be used again within the pressurization chamber.

The depressurized material is then passed to a bioreactor for further processing. The material may be used for a number of different purposes, including methane and ethanol production. A selection of different microorganisms and different conditions, such as aerobic or anaerobic conditions, allows different products to be produced from the biomass. The bioreactor itself may have additional materials, such as trace elements, antifoaming agents, buffers such as calcium carbonate, or growth factors, such as thiamine, added to improve the growth conditions in the bioreactor for the organisms or enzymes used to produce the final products. Other additional materials include, for example, chelators, to avoid the precipitation of metal ions.

The product, such as ethanol or methane, is typically extracted from the bioreactor. This will usually leave a solid waste which may be dried and then burned to produce heat or steam for heating the biomass stream prior to, or during, the pressurization step. Carbon dioxide and/or methane or other gases produced from the bioreactor may also be utilised as the gas using the pressurization step.

The process of the invention improves the efficiency of bioreactors by releasing compounds such as sugars from the biomass stream. It can be used for a wide range of different applications and is especially useful for utilising waste materials and converting them into commercially useful products.

1. A process for treating a eukaryotic cell-derived biomass-containing stream comprising:
passing the stream through a chamber;
pressurizing the stream;
introducing a gas into the pressurized stream, the gas being soluble within the eukaryotic cell-derived biomass; and
depressurizing the stream to cause the solubilised gas to expand and disrupt the eukaryotic cell-derived biomass.

2. A process according to claim 1 wherein the gas comprises carbon dioxide.

3. A process according to claim 1, wherein the eukaryotic cell-derived biomass comprises plant-derived material.

4. A process according to claim 3, wherein the eukaryotic cell-derived biomass comprises cellulose, lignin and/or hemicellulose.

5. A process according to claim 3, wherein the eukaryotic cell-derived biomass comprises wood chippings, paper, sawdust, herbaceous plant material, grass clippings, algae, mixed food materials, cotton, hemp and/or flax.

6. A process according to claim 1, wherein the eukaryotic cell-derived biomass comprises proteinaceous animal material.

7. A process according to claim 6, wherein the proteinaceous animal material comprises collagen, flesh and/or spinal tissue.

8. A process according to claim 1, wherein the eukaryotic cell-derived biomass is within municipal waste.

9. A process according to claim 1, wherein the eukaryotic cell-derived biomass comprises food waste.

10. A process according to claim 1, further comprising treating the stream before and/or during the pressurization step with one or more chemical, physical or biological treatments.

11. A process according to claim 10, wherein the chemical treatment comprises treating with at least one of a wetting agent, an acid, a base, a surfactant, and/or oxidising agent.

12. A process according to claim 10 further comprising heating the stream with steam.

13. A process according to claim 10, wherein the biological treatment comprises the use of one or more enzymes and/or micro-organisms.

14. A process according to claim 1, further comprising the step of passing the depressurized stream into an anaerobic or aerobic bioreactor.

15. A process according to claim 14 wherein the stream is used to produce methanol, ethanol or methane.

16. A process according to claim 14, wherein gas obtained from the bioreactor is collected and introduced into the pressurized stream.

17. A process according to claim 14, wherein the waste material from the bioreactor is dried and burned to directly or indirectly heat the stream.

18. A process according to claim 1, further comprising adding an aqueous liquid to the eukaryotic cell-derived biomass prior to passing the stream through the chamber.

19. An apparatus comprising an entrance port for receiving a eukaryotic cell-derived biomass-containing stream; a port for adding an aqueous liquid to the stream; a chamber for pressurizing the stream, the chamber comprising a port for introducing a gas into the pressurized stream, a depressurization chamber for depressurizing the stream exiting the chamber, and a bioreactor for receiving the depressurized stream.

20. An apparatus for use in a process according to claim 1.

21. (canceled)
